(19) United States
(12) Patent Application Publication
(10) Pub. No.: US 2025/0325740 A1
(43) Pub. Date: Oct. 23, 2025

(54) THORACOSTOMY TUBE WITH INTERNAL BALLOON AND EXTERNAL BOLSTER

(71) Applicant: University of Kentucky Research Foundation, Lexington, KY (US)

(72) Inventors: Brittany E. Levy, Lexington, KY (US); Jennifer T. Castle, Lexington, KY (US)

(21) Appl. No.: 19/187,644

(22) Filed: Apr. 23, 2025

Related U.S. Application Data
(60) Provisional application No. 63/637,798, filed on Apr. 23, 2024.

(51) Int. Cl. A61M 1/00; A61M 1/04; A61M 25/10

(52) U.S. Cl. CPC A61M 1/84; A61M 1/04; A61M 25/1002; A61M 25/1018; A61M 2025/1079; A61M 2205/0205; A61M 2205/0238; A61M 2210/101

(57) ABSTRACT

A thoracostomy tube has a tube body including a lumen, a proximal end, and a distal end. An inflatable internal balloon and an adjustable external bumper are carried on the tube body. When the thoracostomy tube is properly positioned, it is held in place by sandwiching the chest wall between the inflated internal balloon and the external bumper.

### RELATED APPLICATION

This is a utility patent application that claims the benefit of U.S. provisional patent application Ser. No. 63/637,798 filed on Apr. 23, 2024, the full disclosure of which is incorporated herein by reference.

### TECHNICAL FIELD

This document relates to a new and improved thoracostomy tube for draining a hemothorax, pneumothorax, or effusion from the thoracic cavity of a patient as well as to a related method of use.

### BACKGROUND

The pleural cavity is the fluid filled space that surrounds and separates the lungs from surrounding structures including the chest wall, thoracic cage and intercostal spaces, the mediastinum and the diaphragm. A hemothorax is a medical condition characterized by the accumulation of blood in the pleural cavity. A hemothorax can lead to chest pain, difficulty breathing and a rapid heart rate. A pleural effusion is caused by the escape of fluid from the blood vessels or lymphatics into the pleural cavity. A pleural effusion has symptoms similar to a hemothorax. A pneumothorax is characterized by air leaking into and entering the pleural cavity, causing a lung to collapse. A pneumothorax can lead to sharp chest pain and breathing problems.

In order to treat a hemothorax, a pleural effusion or a pneumothorax, it is necessary to release the pressure caused by the blood, air or lymphatic fluid that has improperly entered the pleural cavity. This document relates to a new and improved thoracostomy tube adapted specifically for that purpose. Advantageously, the new thoracostomy tube includes an inflatable balloon and a cooperating external bumper that operate synergistically to (a) decreases the potential for the tube to be pulled out, (b) ensures the sentinel apertures of the tube remain inside the thoracic cavity, and (c) stops movement of the tube body relative to the chest wall, to prevent seeding of external bacteria.

### SUMMARY

In accordance with the purposes and benefits set forth herein, a new and improved thoracostomy tube comprises, consists of or consists essentially of: (a) a tube body having a lumen, a proximal end, and a distal end, (b) an inflatable balloon carried on the tube body, and (c) an adjustable external bolster carried on the tube body between the inflatable balloon and the proximal end of the tube body.

In at least some of the many possible embodiments of the thoracostomy tube, a portion of the tube body distal to the inflatable balloon includes at least one sentinel aperture in communication with the lumen. In some embodiments, the thoracostomy tube further includes a port adapted for inflating and deflating the inflatable balloon. In some of those embodiments, the port is in fluid communication with a dedicated balloon passageway within the tube body.

In at least some embodiments, the thoracostomy tube further includes a radio-opaque strip on the tube body. In at least some embodiments, the radio-opaque strip is carried on the portion of the tube body distal to the inflatable balloon. Such a strip aids in visualizing the proper positioning of the thoracostomy tube, and particularly the distal end of the tube in the pleural cavity of the patient.

In at least some embodiments, the adjustable external bolster has a face oriented toward the inflatable balloon. Preferably, that face includes an antimicrobial coating to aid in preventing a bacterial infection at the incision site where the thoracostomy tube is inserted through the chest wall.

In at least some embodiments, the adjustable external bolster is cone-shaped. In at least some embodiments, the inflatable balloon is donut-shape when inflated. In one particularly useful embodiment, the donut-shaped inflatable balloon has a height of 0.9 to 1.1 centimeters (cm) and a radius of 3.8 to 4.2 cm when inflated.

In accordance with an additional aspect, a related method of draining a hemothorax, pneumothorax, or effusion from a thoracic cavity of a patient, comprises, consists of or consists essentially of:
(a) making an incision on a chest wall of the patient, for example, over top of the fifth rib;
(b) displacing subcutaneous tissue and puncturing a hole through the pleura into a pleural cavity between two ribs of the patient (e.g. over the top of the fifth rib in a fourth-fifth rib space of the patient);
(c) inserting the thoracostomy tube through the hole into the pleural cavity;
(d) inflating the inflatable balloon of the thoracostomy tube within the pleural cavity;
(e) pulling tube slack out of the pleural cavity until the inflated balloon seats against the pleura; and
(f) sliding the external bolster along the tube wall to snug the external bolster against a chest wall of the patient.

The method may also include probing through the hole to ensure a lung is not trapped against the chest wall before the inserting of the thoracostomy tube. Still further, the method may include inserting a finger through the hole to probe and ensure the lung is not trapped against the chest wall.

In the following description, there are shown and described several embodiments of the thoracostomy tube and the related method. As it should be realized, the thoracostomy tube and method are capable of other, different embodiments and their several details are capable of modification is various, obvious aspects all without departing from the apparatus and method as set forth and described in the following claims. Accordingly, the drawings and descriptions should be regarded as illustrative in nature and not as restrictive

### BRIEF DESCRIPTION OF THE DRAWING FIGURES

The accompanying drawing figures incorporated herein and forming a part of the specification, illustrate several aspects of the apparatus and method and together with the description serve to explain certain principles thereof.

FIG. 1 is a perspective view of the new and improved thoracostomy tube.

FIG. 2 is a side elevational view of the new and improved thoracostomy tube also shown in FIG. 1.

FIGS. 3A-3G are a series of views illustrating a method of using the thoracostomy tube of FIGS. 1 and 2.

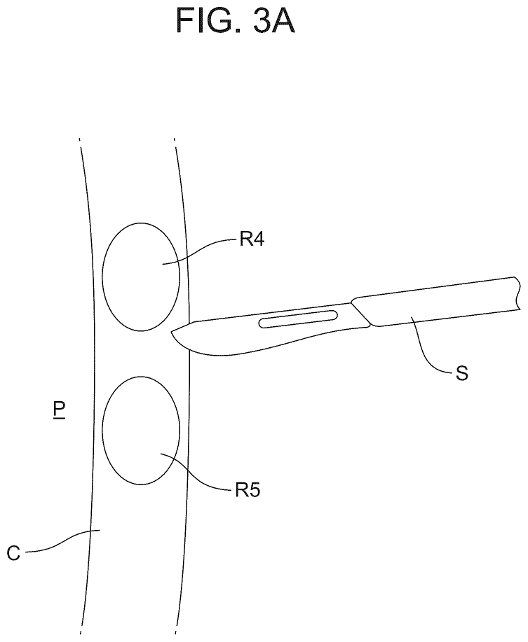

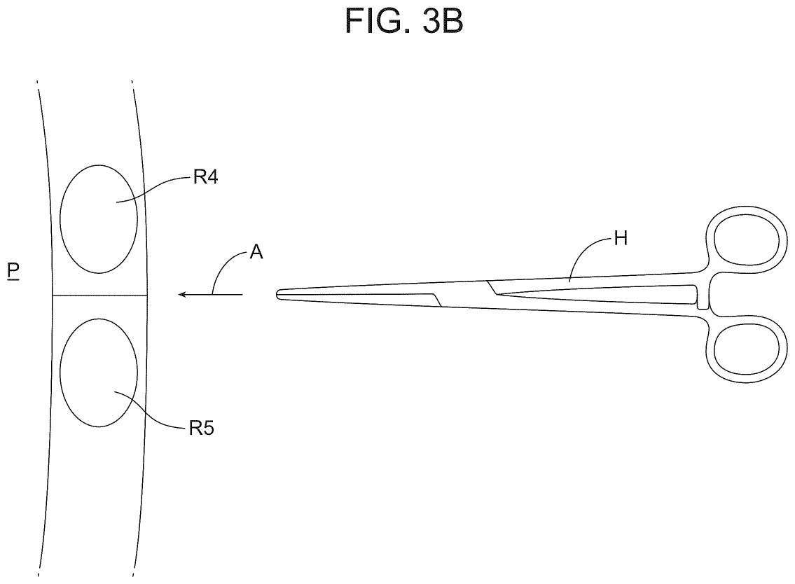

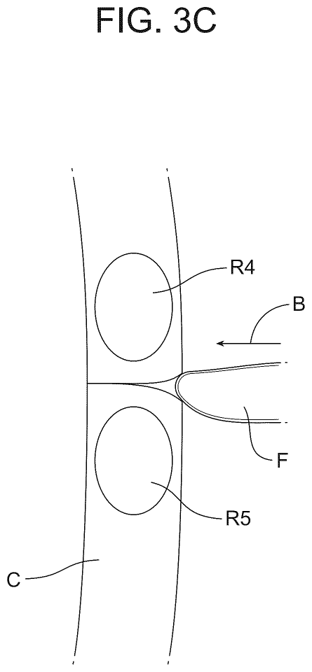

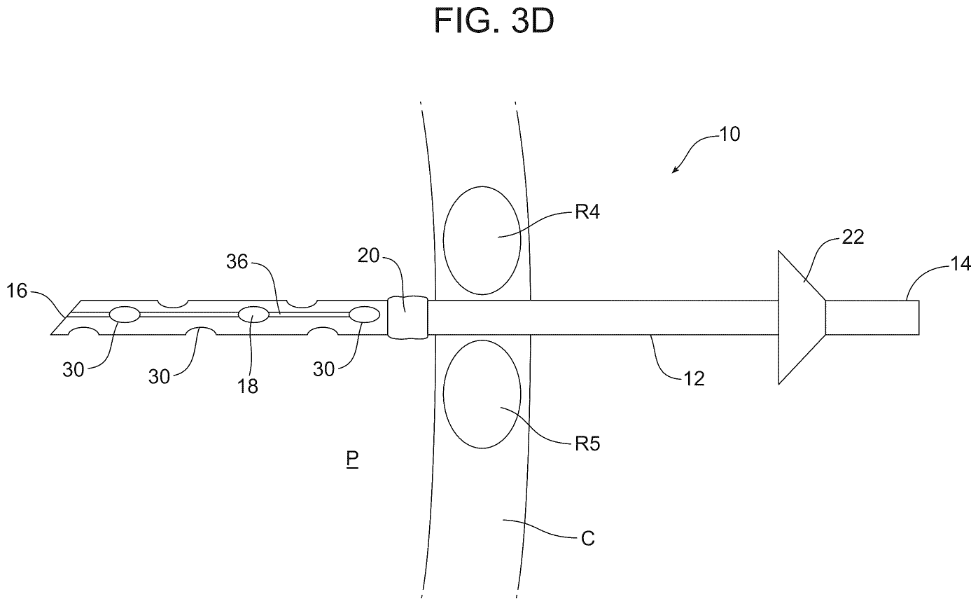

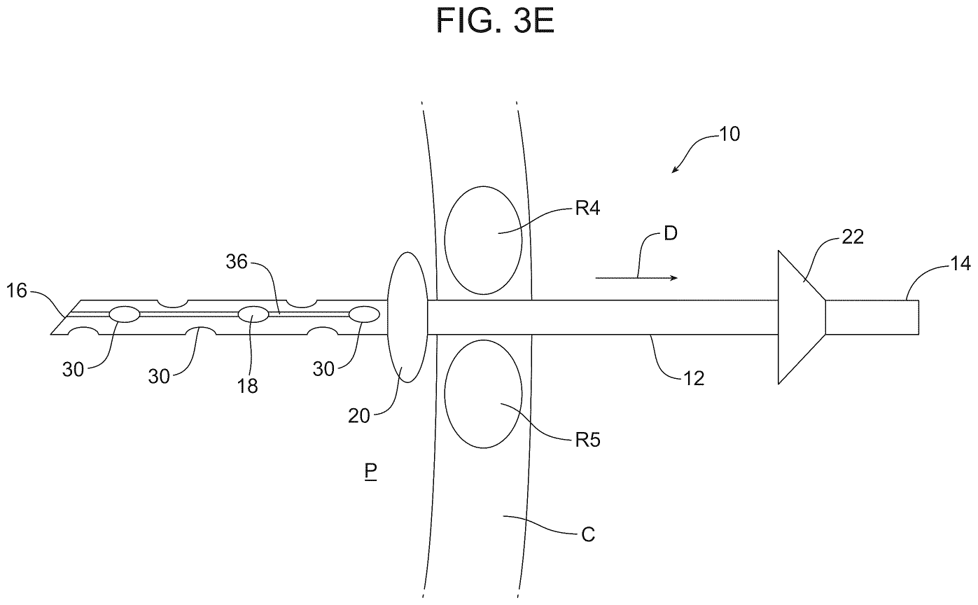

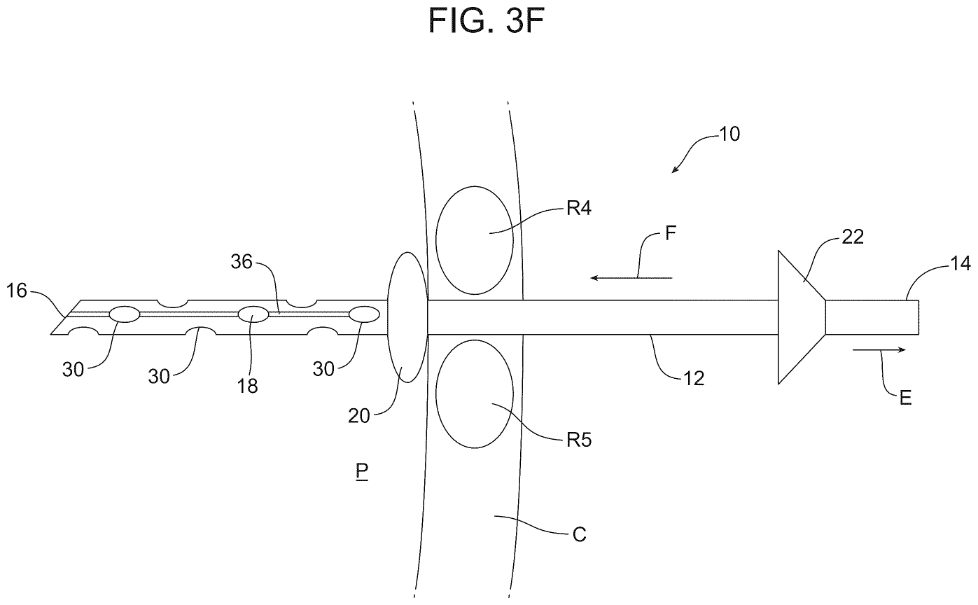

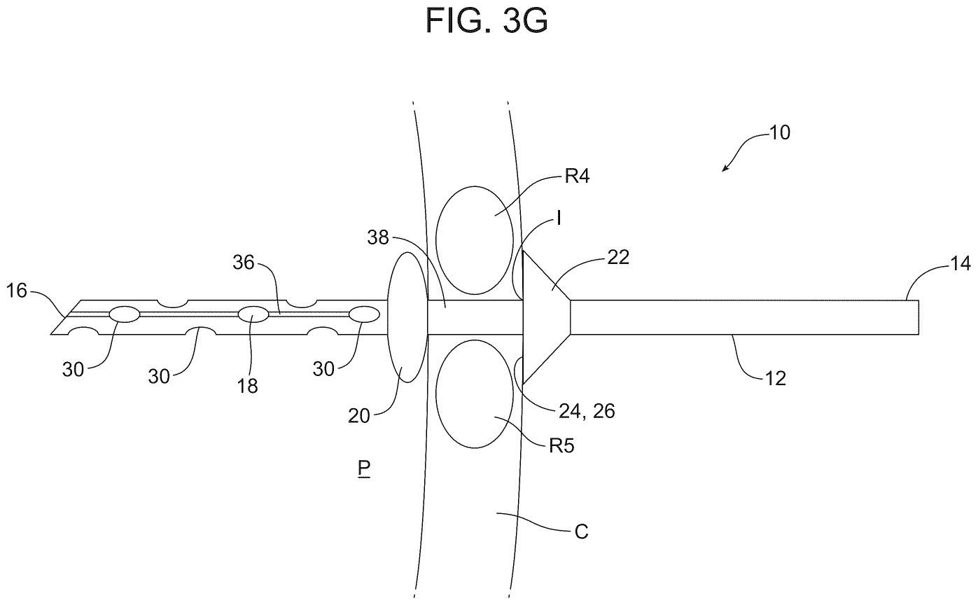

More specifically, FIG. 3A is a schematic view illustrating the making of an incision on a chest wall of the patient over top of a fifth rib. FIG. 3B is a schematic view illustrating the puncturing of a hole through the pleura into the pleural cavity over the top of the fifth rib in a fourth-fifth rib space of the patient with a hemostat. FIG. 3C is a schematic view illustrating the insertion of a finger into the hole to ensure a lung is not trapped against the chest wall. FIG. 3D is a schematic view illustrating the inserting of the thoracostomy tube through the hole into the pleural cavity.

FIG. 3E is a schematic illustration of the thoracostomy tube following insertion and after balloon inflation with an action arrow showing the pulling of tube slack out of the pleural cavity until the inflated balloon seats against the pleura. FIG. 3F is a schematic view illustrating the sliding of the external bumper into position against the chest wall. Finally, FIG. 3G is a schematic illustration showing the thoracostomy tube properly seated for draining the hemothorax, pneumothorax or effusion from the thoracic cavity of the patient.

Reference will now be made in detail to the illustrated embodiments of the apparatus, examples of which are illustrated in the accompanying drawing figures.

### DETAILED DESCRIPTION

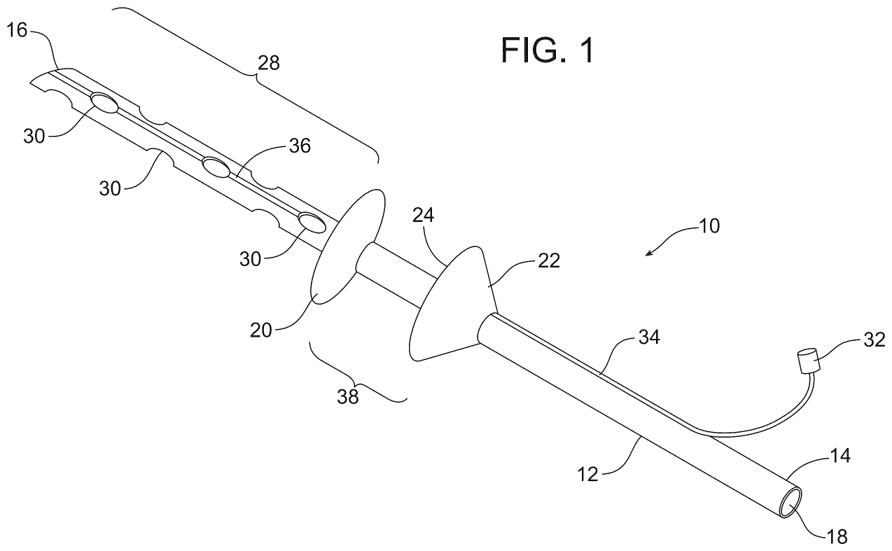

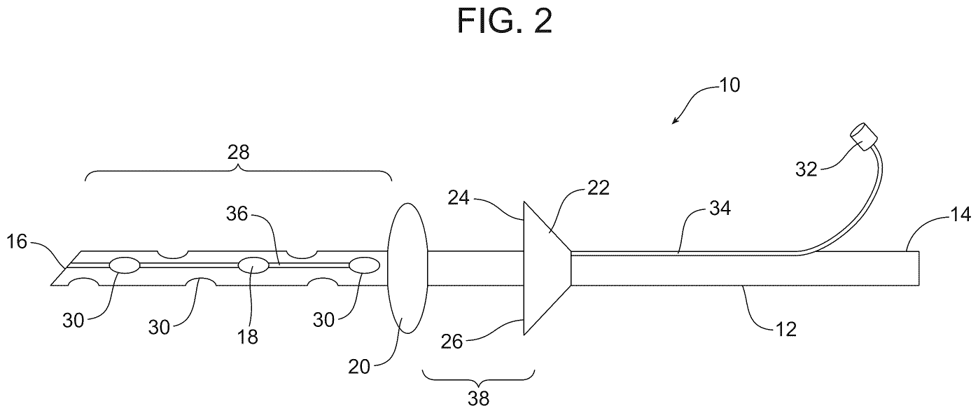

Reference is now made to FIGS. 1 and 2 illustrating the new and improved thoracostomy tube 10 adapted for draining a hemothorax, pneumothorax, or effusion from the thoracic cavity of a patient. As shown, the thoracostomy tube 10 includes a tube body 12 having a proximal end 14 and a distal end 16. The tube body also has a lumen 18 that extends continuously from the proximal end 14 to the distal end 16.

The tube body 12 may be made from any appropriate material known to be useful for such medical purposes. For example, the tube body 12 may be made from sterilizable medical plastics. In one particularly useful embodiment, the tube body 12 is made from polyvinyl chloride (PVC) or silicone. The tube body 12 may have any appropriate diameter. In some embodiments, the tube body 12 has a diameter of between 6 French and 40 French.

The tube body 12 carries an internal, inflatable balloon 20 and an adjustable external bolster 22. The inflatable balloon 20 may also be made from PVC or silicone as well as other appropriate materials. The balloon 20, when inflated, may assume any appropriate shape and size. In one particularly useful embodiment, the balloon 20 is donut-shape when inflated. When fully inflated, the donut-shaped inflatable balloon 20 may have a height of 0.9 to 1.1 cm and a radius of 3.8 to 4.2 cm.

The adjustable external bolster 22 may be made of any appropriate material including, but not limited to, PVC and silicone. The adjustable external bolster 22 fits snuggly around the tube body 12 and will hold its position relative to the tube body, but allows a user to apply positive pressure to selectively slide the adjustable external bolster along and relative to the tube body in a manner described in greater detail below. The external bolster 22 has a face 24 oriented toward the inflatable balloon 20. This face 24 may include an antimicrobial coating 26. Such an antimicrobial coating may be made from any material known in the art to be useful for such a purpose. Such materials include, but are not limited to, silver nanoparticles, quaternary ammonium salts, cationic polymers such as cationic pyridinium, copper nanoparticles, and combinations thereof.

The portion 28 of the tube body 12 distal to the internal inflatable balloon 20 includes one or more sentinel apertures 30 in communication with the internal lumen 18 in the tube body. The sentinel apertures 30 may be of any desired shape such as round or oval. The size of each sentinel aperture 30 may range from about 3 to 5 square millimeters (mm). The sentinel openings 30 may all be of the same or different sizes and shapes.

A port 32 may be provided near the proximal end 14 of the tube body 12. The port 32 allows one to inflate or deflate the balloon 20 as desired through the dedicated balloon passageway 34. In the illustrated embodiment, that passageway 34 is formed in the wall of the tube body 12 outside of the lumen 18. A radio-opaque strip 36 may be provided on the distal end 16 of the tube body 12: that is, distal to the balloon 20. The radio-opaque strip is adapted to allow precise tracking and placement of the distal end 16 of the tube body 12 during X-ray monitoring or other imaging in a manner known in the art.

The portion of the tube body 12 between the internal inflatable balloon 14 and the adjustable external bolster 16 is referred to as the intrathoracic portion 38. When the thoracostomy tube 10 is properly positioned or seated, it is the intrathoracic portion 38 that extends through the chest wall of the patient.

In use, first an incision of about 2-4 cm is made with a scalpel S on the chest wall C. In the illustrated embodiment, that incision is made over top the 5^{th} rib R5. See FIG. 3A. Using hemostats H, the subcutaneous tissue is displaced, and a hole is punctured into the pleural cavity P over top the 5^{th} rib, in the 4-5^{th} rib spaces. See FIG. 3B and note action arrow A. Next a finger F is inserted through the hole into the thoracostomy to ensure the lung is not trapped against the internal chest wall. This is termed a finger thoracostomy. See FIG. 3C and note action arrow B. Finally the thoracostomy tube 10 is inserted into the hole. See FIG. 3D. The balloon 20 remains uninflated during this insertion.

Once the balloon 20 is fully inserted into the pleural cavity P (this may be confirmed by radio-opaque markings (not shown) on the balloon), the balloon 20 is inflated, and the tube slack is pulled out of the pleural cavity P (note action arrow D in FIG. 3E) until the balloon is seated against the internal chest wall C. Next, the external bolster 22 is adjusted by sliding along the tube body 12. While a slight pressure is maintained upon the tube body 12 to hold the inflated balloon 20 against the interior wall of the pleural cavity P (note action arrow E in FIG. 3F), the external bolster 22 is snugged into place against the chest wall W (note action arrow F in FIG. 3F): that is, one slides the bolster 22 along the tube body 12. In contrast, in state of the art medical practice, after the tube is inserted, it is sutured into place in a Greek sandal configuration. There is no external bolster 22. FIG. 3G illustrates the thoracotomy tube 10 properly placed within the pleural cavity P of a patient.

Once the tube 10 has been properly seated, as shown and described above, the proximal end 14 of the tube body 12 is connected to a pleuravac drainage system of a type known in the art in order to draw fluid pressure from the pleural cavity P and relieve the chest pain of the patient.

Advantageously, the thoracostomy tube 10 disclosed herein, includes an internal inflated balloon bumper 20 and an external bolster or bumper 22 that are snugged against both sides of the chest wall C, effectively sandwiching the chest wall and decreasing the potential for the tube 10 to be pulled out. The internal balloon 20 ensures the distal portion 28 of the tube 10, including the sentinel apertures 30, remains inside the thoracic cavity C to allow fluid pressure to be relieved. In addition, the external bolster or bumper 22 stops movement of the tube body 12 relative to the chest wall C, to prevent seeding of external bacteria. The antimicrobial coating 26 against the tube incision I aids in resisting or preventing infection.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “an aperture”, as used herein, may also refer to, and encompass, a plurality of apertures.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified. Terms of approximation, such as the terms about, substantially, approximately, etc., as used herein, refers to ±10% of the stated numerical value.

Although the thoracostomy tube 10 and related method of use of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A thoracostomy tube, comprising:
a tube body having a lumen, a proximal end, and a distal end;
an inflatable balloon carried on the tube body; and
an adjustable external bolster carried on the tube body between the inflatable balloon and the proximal end of the tube body.

2. The thoracostomy tube of claim 1, wherein a portion of the tube body distal to the inflatable balloon includes at least one sentinel aperture in communication with the lumen.

3. The thoracostomy tube of claim 2, further including a port adapted for inflating and deflating the inflatable balloon.

4. The thoracostomy tube of claim 3, wherein the port is in fluid communication with a dedicated balloon passageway within the tube body.

5. The thoracostomy tube of claim 3, further including a radio-opaque strip on the tube body.

6. The thoracostomy tube of claim 5, wherein the radio-opaque strip is carried on the portion of the tube body distal to the inflatable balloon.

7. The thoracostomy tube of claim 2 wherein the adjustable external bolster has a face oriented toward the inflatable balloon.

8. The thoracostomy tube of claim 7, wherein the face includes an antimicrobial coating.

9. The thoracostomy tube of claim 8, wherein the adjustable external bolster is cone-shaped.

10. The thoracostomy tube of claim 9, wherein the inflatable balloon is donut-shape when inflated.

11. The thoracostomy tube of claim 10, wherein the donut-shaped inflatable balloon has a height of 0.9 to 1.1 cm and a radius of 3.8 to 4.2 cm when inflated.

12. A method of draining a hemothorax, pneumothorax or effusion from a thoracic cavity of a patient, comprising:
making an incision on a chest wall of the patient;
displacing subcutaneous tissue and puncturing a hole through a pleura into a pleural cavity in a space between two ribs of the patient;
inserting the thoracostomy tube of claim 1 through the hole into the pleural cavity;
inflating the inflatable balloon of the thoracostomy tube within the pleural cavity;
pulling tube slack out of the pleural cavity until the inflated balloon seats against the pleura; and
sliding the external bolster along the tube wall to snug the external bolster against a chest wall of the patient.

13. The method of claim 12, further including probing through the hole to ensure a lung is not trapped against the chest wall before the inserting of the thoracostomy tube.

14. The method of claim 13, including inserting a finger through the hole to probe and ensure the lung is not trapped against the chest wall.
